(19) United States
(12) Patent Application Publication
(10) Pub. No.: US 2024/0010628 A1
(43) Pub. Date: Jan. 11, 2024

(54) METHODS OF MANUFACTURE OF R-MDMA

(71) Applicant: Mind Medicine, Inc., New York, NY (US)

(72) Inventors: Stephen E. SCHNEIDER, Raleigh, NC (US); Derek LONDESBROUGH, Hartlepool (GB)

(73) Assignee: Mind Medicine, Inc., New York, NY (US)

(21) Appl. No.: 18/344,584

(22) Filed: Jun. 29, 2023

Related U.S. Application Data
(60) Provisional application No. 63/359,712, filed on Jul. 8, 2022.

(51) Int. Cl. C07D 317/58

(52) U.S. Cl. CPC C07D 317/58

(57) ABSTRACT

A method of manufacturing R-MDMA by forming a Grignard reagent from 5-bromobenzodioxole, treating the Grignard reagent with S-propylene oxide to form chirally pure alcohol 1, activating the alcohol as mesylate 2, converting to chirally pure azide 3, reducing the azide to amine 4, protecting the amine with di-tert-butyl dicarbonate, reducing the protected amine 5 to yield R-MDMA free base 6, and treating with an acid to form a salt 7 in >99% e.e. A method of manufacturing S-MDMA by forming a Grignard reagent from 5-bromobenzodioxole, treating the Grignard reagent with R-propylene oxide to form chirally pure alcohol 8, activating the alcohol as mesylate 9, converting to chirally pure azide 10, reducing the azide to amine 11, protecting the amine with di-tert-butyl dicarbonate, reducing the protected amine 12 to yield S-MDMA free base 13, and treating with an acid to form a salt 14 in >99% e.e.

### BACKGROUND OF THE INVENTION

### 1. Technical Field

The present invention relates to methods for manufacturing R-MDMA.

### 2. Background Art

3,4-Methylenedioxymethamphetamine (MDMA) is a psychoactive drug that alters mood and perception, and is investigated as an adjunct in psychotherapy for posttraumatic stress disorder (PTSD), social anxiety, autism (Danforth, 2016; Danforth et al., 2018; Danforth et al., 2016; Mithoefer et al., 2019; Mithoefer et al., 2010; Oehen et al., 2013), and may later also be studied and used for a range of other medical conditions. Such conditions where MDMA or related substances may be useful include, but are not limited to, substance-use disorder, depression, anxiety disorder (including social anxiety), anxiety with life-threatening disease, personality disorder including narcistic and antisocial disorder, autism and other developmental disorders and obsessive-compulsive disorder. MDMA or related substances can also be used to enhance individual or couple therapy.

There are several side effects and safety concerns regarding MDMA. Abuse of MDMA can produce hyperpyrexia, neurocognitive defects, and increased rates of depression. MDMA can also be neurotoxic which limits its ability to be used chronically with repeat administration. Use of MDMA often impairs declarative memory, prospective memory, and higher cognitive skills. Neurocognitive deficits are associated with reduced SERT in the hippocampus, parietal cortex, and prefrontal cortex. EEG and ERP studies have shown localized reductions in brain activity during neurocognitive performance. Deficits in sleep, mood, vision, pain, psychomotor skill, tremor, neurohormonal activity, and psychiatric status, have also been demonstrated. These effects are seen more with higher doses or longer use. (Parrott, Neuroscience & Biobehavioral Reviews, Volume 37, Issue 8, 2013, Pages 1466-1484).

MDMA has two enantiomers, S(+)-MDMA and R(−)-MDMA. The R enantiomer is thought to be more active (Nichols, et al. J. Med. Chem. 1986, 29, 2009-2015). It is believed that the neurotoxicity of racemic MDMA is caused by the S(+) enantiomer, not the R(−) enantiomer due to the low efficacy of the R(−) enantiomer as a releaser of dopamine. The R(−) enantiomer also does not produce hyperthermia. The R(−) enantiomer may have a lower risk of abuse. (Pitts, et al. Psychopharmacology (2018) 235:377-392). It has been shown that the enantiomers have different effects. R-MDMA and S-MDMA were evaluated for their effects in a parkinsonian animal model (Huot, et al., The Journal of Neuroscience, May 11, 2011, 31(19):7190-7198), and it was found that R-MDMA, which is a selective compound for 5-HT2A receptors, decreased severity of peak-dose dyskinesia and increased duration of good ON-time, S-MDMA, which exhibits high affinity for SERT and moderate affinity for DAT, extended total duration of ON-time but exacerbated dyskinesia. This showed that racemic MDMA exerts simultaneous effects, reducing dyskinesia and extending ON-time, by 5-HT2A antagonism and SERT-selective mixed monoamine uptake inhibition, which arise from its R and S enantiomers, respectively. Therefore, it can be advantageous to use R-MDMA in treatments.

In order to use R-MDMA in treatments, it must first be obtained efficiently and in a substantially pure form. Chiral resolution of racemic MDMA has been performed to yield R-MDMA (Taschwer, Magdalena; Seidl, Yvonne; Mohr, Stefan; Schmid, Martin G., Chirality, 2014, vol. 26, #8, p. 411-418). Enantioselective synthesis of R-MDMA has been performed, shown in FIG. 1 (D. E. Nichols, A. J. Hoffman, R. A. Oberlender, P. Jacob, A. T. Shulgin, Derivatives of 1-(1,3-benzodioxol-5-yl)-2-butanamine: representatives of a novel therapeutic class, J. Med. Chem. 29 (1986) 2009-2015.). This process requires 8 steps to get to compound 2a with a 23% overall yield. S. Llabres et al. (European Journal of Medicinal Chemistry 81 (2014) 35-46) teaches a process with 7 steps and 32% overall yield to free base on 100 mg scale final product with 99% enantiomeric excess (e.e.). The process utilized nitromethane and 2 silica purifications. Neajdenko, et al. teaches another process shown in FIG. 2 (The Journal of Neuroscience, May 11, 2011, 31(19):7190-7198). However, each of these processes provide low yields, use harsh reagents and/or conditions, and/or require chromatographic purifications that are not suitable for large scale pharmaceutical use.

Therefore, there remains a need for an efficient, robust, scalable synthesis of R-MDMA for pharmaceutical development capable of delivering >99% e.e.

### SUMMARY OF THE INVENTION

The present invention provides for a method of manufacturing R-MDMA, as shown in FIG. 3, by forming a Grignard reagent from 5-bromobenzodioxole, treating the Grignard reagent with S-propylene oxide to form chirally pure 1 ((S)-1-(benzo[d][1,3]dioxol-5-yl)propan-2-ol), activating the alcohol as mesylate 2 ((S)-1-(benzo[d][1,3]dioxol-5-yl)propan-2-yl methanesulfonate), converting to chirally pure azide 3 ((R)-5-(2-azidopropyl)benzo[d][1,3]dioxole), reducing the azide to amine 4 ((R)-1-(benzo[d][1,3]dioxol-5-yl)propan-2-amine), protecting the amine with di-tert-butyl dicarbonate to form protected amine 5 (ethyl (R)-(1-(benzo[d][1,3]dioxol-5-yl)propan-2-yl)carbamate), reducing the protected amine 5 to yield R-MDMA free base 6 ((R)-1-(benzo[d][1,3]dioxol-5-yl)-N-methylpropan-2-amine), and treating with an acid to form a salt, such as 7 (7-(R)-1-(benzo[d][1,3]dioxol-5-yl)-N-methylpropan-2-amine hydrochloride) in >99% e.e.

The present invention provides for a method of manufacturing S-MDMA, as shown in FIG. 4, by forming a Grignard reagent from 5-bromobenzodioxole, treating the Grignard reagent with R-propylene oxide to form chirally pure 8 ((R)-1-(benzo[d][1,3]dioxol-5-yl)propan-2-ol), activating the alcohol as mesylate 9 ((R)-1-(benzo[d][1,3]dioxol-5-yl)propan-2-yl methanesulfonate), converting to chirally pure azide 10 ((S)-5-(2-azidopropyl)benzo[d][1,3]dioxole), reducing the azide to amine 11 ((S)-1-(benzo[d][1,3]dioxol-5-yl)propan-2-amine), protecting the amine with di-tert-butyl dicarbonate to form a protected amine 12 (ethyl (S)-(1-(benzo[d][1,3]dioxol-5-yl)propan-2-yl)carbamate), reducing the protected amine 12 to yield S-MDMA free base 13 ((S)-1-(benzo[d][1,3]dioxol-5-yl)-N-methylpropan-2-amine), and treating with an acid to form a salt such as 14 ((S)-1-(benzo[d][1,3]dioxol-5-yl)-N-methylpropan-2-amine hydrochloride) in >99% e.e.

### DESCRIPTION OF THE DRAWINGS

Other advantages of the present invention are readily appreciated as the same becomes better understood by reference to the following detailed description when considered in connection with the accompanying drawings wherein:

FIG. 1 is a reaction schematic of the prior art;

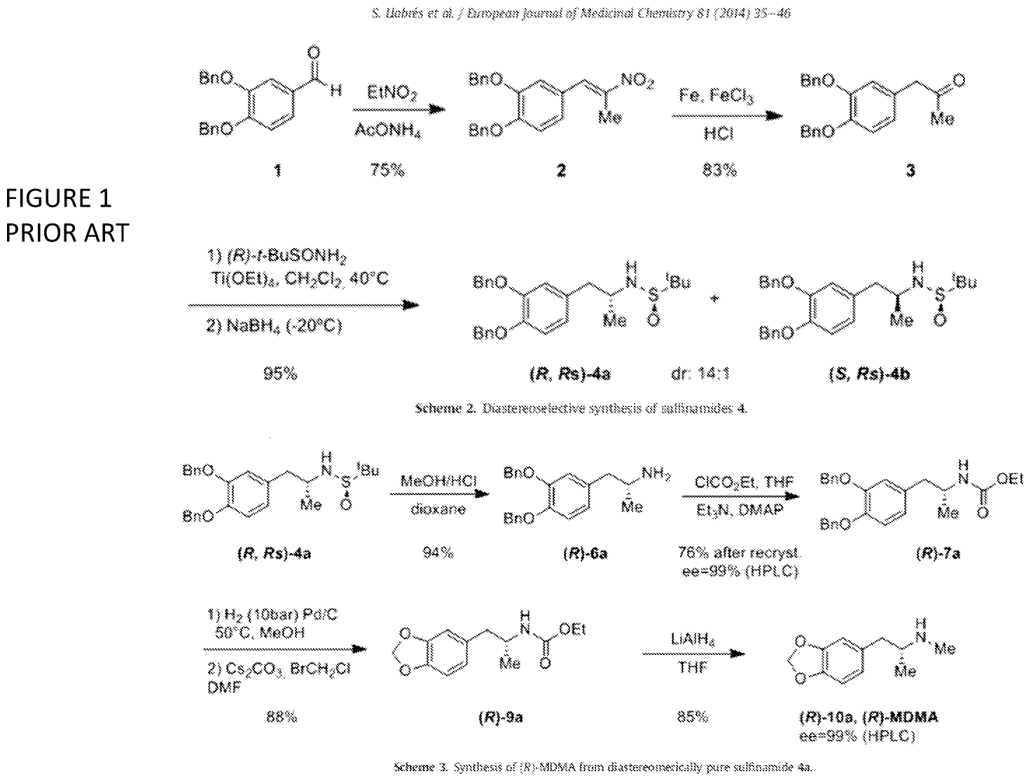

FIG. 2 is a reaction schematic of the prior art;

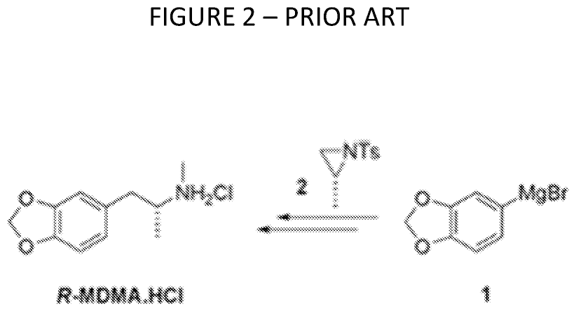

FIG. 3 is a reaction schematic for producing R-MDMA of the present invention;

FIG. 4 is a reaction schematic for producing S-MDMA of the present invention;

FIG. 5 is a HPLC chromatogram of recrystallized R-MDMA HCl;

FIG. 6 is a chiral HPLC chromatogram of recrystallized R-MDMA HCl;

FIG. 7 is a ¹H NMR spectrum of recrystallized R-MDMA HCl;

FIG. 8 is a combined DSC/TGA of recrystallized R-MDMA HCl;

FIG. 9 is a HPLC chromatogram of S-MDMA;

FIG. 10 is a chiral HPLC chromatogram of S-MDMA; and

FIG. 11 is a ¹H NMR spectrum of S-MDMA.

### DETAILED DESCRIPTION OF THE INVENTION

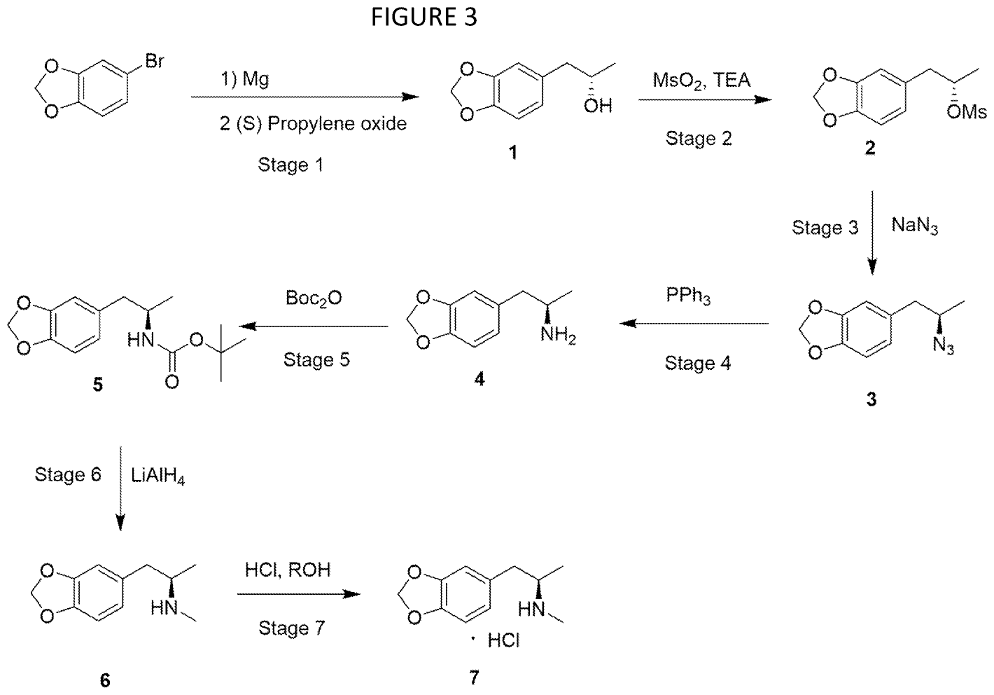

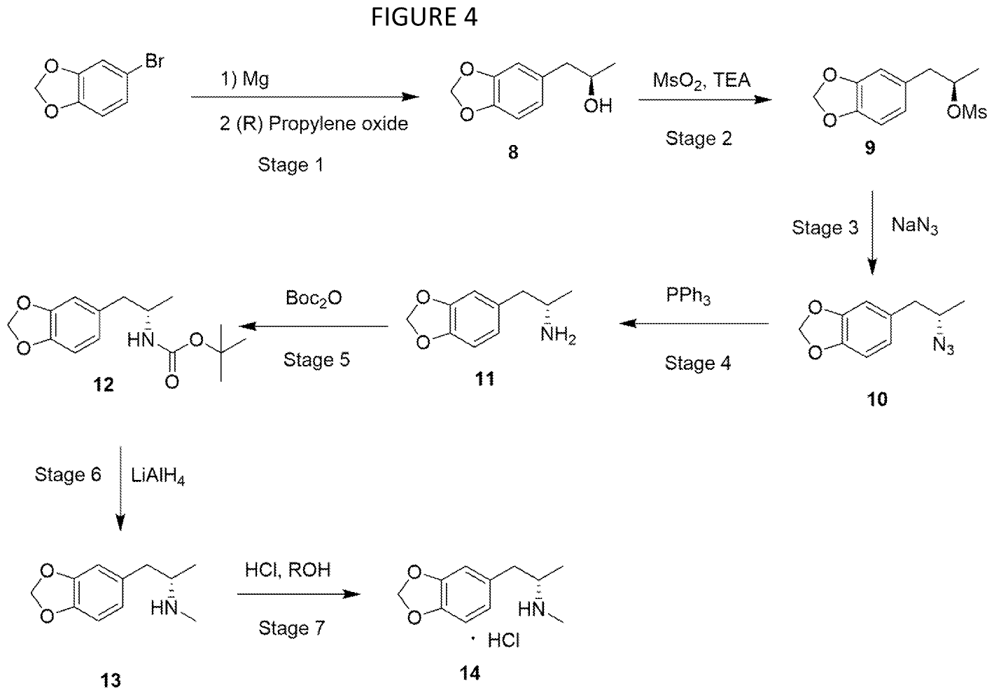

The present invention provides for a method of manufacturing and synthesizing R-MDMA or S-MDMA, shown generally for R-MDMA in FIG. 3 and for S-MDMA in FIG. 4. This process uses 7 steps with a greater than 30% overall yield on a 2 kg scale.

In general, a Grignard reagent is formed from 5-bromobenzodioxole which is then treated with S-propylene oxide to form chirally pure 1 ((S)-1-(benzo[d][1,3]dioxol-5-yl)propan-2-ol). The alcohol is activated as the mesylate 2 ((S)-1-(benzo[d][1,3]dioxol-5-yl)propan-2-yl methanesulfonate) and then converted to chirally pure 3 ((R)-5-(2-azidopropyl)benzo[d][1,3]dioxole). The azide is reduced to 4 ((R)-1-(benzo[d][1,3]dioxol-5-yl)propan-2-amine) and the resulting amine is protected with di-tert-butyl decarbonate yielding 5 (ethyl (R)-(1-(benzo[d][1,3]dioxol-5-yl)propan-2-yl)carbamate). The Boc-protected amine is reduced to yield R-MDMA free base (6) ((R)-1-(benzo[d][1,3]dioxol-5-yl)-N-methylpropan-2-amine) with is treated with acid to form a salt (7) (such as (R)-1-(benzo[d][1,3]dioxol-5-yl)-N-methylpropan-2-amine hydrochloride) in >99% e.e. Applicants have achieved >99.9% e.e. on >100 g scale.

This method has several advantages over the prior art. This is a scalable process that utilizes standard pharmaceutical manufacturing reagents and processes and commercially available starting materials. Chirality is introduced in the first step and chiral purity is maintained at >99% throughout the synthesis. There are minimal process impurities, and the product is >99A % pure without the need for any chromatography or recrystallizations, though recrystallization may be employed, if desired. The process does not use ICH Class 1 solvents or ICH Class 1 or 2 metal catalysts. Though sodium azide is utilized in the process, this reagent and the azide intermediate can be handled safely by those experienced in this area. MsCl can be used instead of MsO₂ in Stage 2. The amine resulting from Stage 4 can be converted to a salt if needed for the purpose of purification or storage. Other protecting groups (i.e., ethyl chloroformate) can be used instead of Boc₂O in Stage 5, though Boc₂O is preferred and Fmoc-CI and Benzyl chloroformate did not give acceptable results. Reducing agents other than LiAlH₄ (lithium aluminium hydride) can be used in Stage 6.

Alternate salts can be produced by exchanging other acids for HCl to obtain hydrobromide, maleate, L-malate, D-tartrate, meso-tartrate, citrate, phosphate, naphthylene-1,5-disulphonate, fumarate, sulfate, mesylate, acetate, or oxalate salts. In other words, the acids that can be used include, but are not limited to, HCl, HBr, D-tartaric acid, L-tartaric acid, meso-tartaric acid, oxalic acid, maleic acid, malic acid, citric acid, phosphoric acid, napthylene-1,5-disulphonic acid, furmaric acid, sulfuric acid, methanesulphonic acid, acetic acid, or oxalic acid. S-MDMA can be produced by the same process by exchanging S-propylene oxide for R-propylene oxide in Stage 1, shown in FIG. 4. Therefore a method of manufacturing S-MDMA is also provided by forming a Grignard reagent from 5-bromobenzodioxole, treating the Grignard reagent with R-propylene oxide to form chirally pure 8 ((R)-1-(benzo[d][1,3]dioxol-5-yl)propan-2-ol), activating the alcohol as mesylate 9 ((R)-1-(benzo[d][1,3]dioxol-5-yl)propan-2-yl methanesulfonate), converting to chirally pure azide 10 ((S)-5-(2-azidopropyl)benzo[d][1,3]dioxole), reducing the azide to amine 11 ((S)-1-(benzo[d][1,3]dioxol-5-yl)propan-2-amine), protecting the amine with di-tert-butyl decarbonate to yield 12 (ethyl (S)-(1-(benzo[d][1,3]dioxol-5-yl)propan-2-yl)carbamate), reducing the protected amine to yield S-MDMA free base 13 ((S)-1-(benzo[d][1,3]dioxol-5-yl)-N-methylpropan-2-amine), and treating with an acid to form a salt 14 (such as (S)-1-(benzo[d][1,3]dioxol-5-yl)-N-methylpropan-2-amine hydrochloride) in >99% e.e.

The invention is further described in detail by reference to the following experimental examples. These examples are provided for the purpose of illustration only, and are not intended to be limiting unless otherwise specified. Thus, the invention should in no way be construed as being limited to the following examples, but rather should be construed to encompass any and all variations which become evident as a result of the teaching provided herein.

### Example 1

### Stage 1—Preparation of (S)-1-(benzo[d][1,3]dioxol-5-yl)propan-2-ol (1)

Magnesium turnings (948 g, 39.0 mol, 1.1 eq) and THF (28.5 L) were mixed and heated to reflux. 5-Bromo-1,3-benzodioxle (142 g, 0.71 mol, 0.02 eq) was charged in one portion. Previously prepared Grignard reagent (356.3 ml) was charged in one portion and initiation was observed after 10 min. The remaining 5-bromo-1,3-benzodioxole (6983 g, 0.98 eq, 34.74 mol) was charged dropwise over 1.5 hours while maintaining a temperature of 65-75° C. and the mixture was stirred at reflux for 30 minutes. The mixture was cooled to 5° C. over 50 minutes then stirred overnight at 5° C. Copper iodide (114.7 g, 0.60 mol, 0.02 eq) was charged in one portion. (S)-Propylene oxide (2235 ml, 33.10 mol, 0.9 eq) dissolved in THF (2235 ml) was charged dropwise at ˜ 1.5 L/hr (3 hours total, T<10° C.). The reaction was stirred at 5° C. for 30 minutes until complete by HPLC. The contents were worked-up in two equal portions.

Split Work-Up (First Portion)

To a second jacketed vessel was charged acetic acid (1220 ml) then 10% brine (4.8 L). Half of the reaction mass was transferred to the second vessel while maintaining T<40° C. The contents were stirred at 40° C. for 1 hour then left for the phases to separate overnight. The lower aqueous phase was removed from the vessel retaining the upper organic in the vessel. heptane (7.3 L) and 10% brine (7.1 L) were charged in one portion and stirred at 30° C. for 40 minutes. The phases were separated from the vessel. The upper organic layer was filtered and washed with heptane (2 L).

The second portion was worked up following the same procedure as the first portion. The organics from each work-up portion were combined and concentrated to give a crude oil.

WFE Distillation

The crude oil was diluted with PEG400 (800 ml) and distilled via a wiped film evaporator (three passes were performed). Each pass was analyzed by HPLC, chiral LC and NMR. Pass 1: HPLC 92.9%, NMR assay (CDCl₃) 95%, chiral purity 99.4%, active mass=3384 g. Pass 2: HPLC 96.3%, NMR assay 99%, chiral purity 99.4%, active mass=1598 g. Pass 3: HPLC 98.0%, NMR assay 97%, chiral purity 99.6%, 99.2% e.e., active mass=231.3 g. Overall yield=5414 g (5214 g active, 80%).

### Example 2

### Stage 2—Preparation of (S)-1-(benzo[d][1,3]dioxol-5-yl)propan-2-yl methanesulfonate (2)

To a reactor were charged 1 (1300.8 g, 95% activity, 6.86 mol, 1 eq) and EtOAc (12.4 L) giving a clear solution. Methanesulfonic anhydride (1800 g, 10.33 mol, 1.2 eq) was charged portion wise over 10 minutes. The contents were cooled to 6° C. and triethylamine (2.04 L, 14.63 mol, 1.5 eq) was charged dropwise over 3 hours. The contents were sampled by HPLC after 20 minutes. IPC 1: 0.8% 1, 98.5% 2. Water (3.7 L) was charged in one portion and stirred for 10 minutes. The stirrer was stopped to allow the phases to settle over 5 minutes. The lower aqueous phase was removed via vacuum transfer. Water (3.7 L) was charged in one portion and stirred for 10 minutes. The stirrer was stopped to allow the phases to settle over 10 minutes. The lower aqueous phase was removed via vacuum transfer. 10% Brine solution (3.7 L, equivalent to 1 kg of NaCl dissolved into 9 L of water) was charged and stirred for 10 minutes. The stirrer was stopped to allow the phases to settle and the layers were separated. The organic phases were combined, dried over magnesium sulfate, filtered, and concentrated. 2 was isolated as a brown oil, 3791.3 g. HPLC: 88.9%. ¹H NMR (CDCl₃): conforms to structure.

### Example 3

### Stages 3/4—Preparation of (R)-1-(benzo[d][1,3]dioxol-5-yl)propan-2-amine (4)

To a reactor were charged stage 2 (770 g crude mass, 81% activity, 2.41 mol, 1 eq) and DMF (2.7 L). Sodium azide (230.2 g, 35.54, 1.4 eq) was charged in one portion. The contents were heated to 60° C. over 45 minutes and stirred overnight. The contents were cooled to 15° C. and water (3.4 L) was charged dropwise over 45 minutes. The phases were separated and the aqueous phase was treated with TBME (1.35 L). The phases were allowed to settle and the aqueous was removed. The organic layers were combined and water (3.4 L) was charged. The phases were allowed to settle and the lower layer was removed. Saturated sodium bicarbonate solution (3.4 L) was charged and stirred. The stirring was stopped, and the phases were allowed to settle over 10 minutes. The lower layer was removed and fresh water was charged to the vessel. The phases were allowed to settle and separated. The organic layer was washed with water (3.4 L) and the aqueous layer was removed. Analysis of the organic layer showed <129 ppm NaN₃. THF (676 ml) and water (1.35 L) were added. The contents were heated to 40° C. over 20 minutes and triphenyl phosphine (726 g, 2.77 mol, 1.15 eq) was charged portion wise over 1 hour. The contents were stirred cooling to 15° C. after 24 hours. Analysis showed 87.5% 4 with no 3 remaining. Water (1.9 L) was charged in one portion followed by concentrated HCl (250 ml) dropwise over 40 minutes. The phases were separated and the aqueous was washed with iso-propyl acetate (3×1.9 L). 85% KOH (315 g) was charged portion wise over 15 minutes. The aqueous phase was extracted with MTBE (3×1.9 L). The organics were combined, dried over MgSO₄, filtered, and concentrated to yield 4 as a brown oil=383.6 g. HPLC: 98.8%. Chiral purity: 99.3%, 98.6% e.e. NMR assay (CDCl₃): 96%, Active yield=368.3 g (85% yield).

### Example 4

### Stage 5—Preparation of ethyl (R)-(1-(benzo[d][1,3]dioxol-5-yl)propan-2-yl)carbamate (5)

To a reactor were charged 4 (1691 g crude, 95% activity, 8.93 mol, 1 eq) and THF (4 L). The resulting solution was cooled to 0° C. Boc-anhydride (2065 g, 9.46 mol, 1.05 eq) dissolved into THF (4 L) was charged dropwise and the contents were heated to RT and stirred until the reaction was complete by HPLC. The crude reaction was concentrated via rotavapor to afford a beige solid which was dried at 40° C. to yield 2297.6 g. HPLC: 98.9 area %. NMR assay (CDCl₃): 97%. Active yield=2228.7 g (89% yield).

### Example 5

### Stage 6—Preparation of (R)-1-(benzo[d][1,3]dioxol-5-yl)-N-methylpropan-2-amine (6)

To a reactor were charged 5 (572 g, 97% activity, 1.99 mol, 1 eq) and THF (1.1 L). The resulting solution was cooled to 10° C. and 1 M LiAlH₄ (4.44 L, 4.44 mol, 2.2 eq) was charged dropwise. The contents were heated to 40° C. and stirred until complete by HPLC. MTBE (2.78 L) was charged in one portion and the contents were cooled to 0° C. Water (166 ml) was charged dropwise over 1 hour. 15% NaOH (166 ml, equivalent to dissolving 150 g NaOH in 850 ml water) was charged dropwise over 20 min. Water (500 ml) was charged dropwise over 15 minutes. The contents were heated to 20° C. and stirred for 2 hours. Magnesium sulphate (677 g) was charged portion wise over 10 minutes: +5° C. exotherm. The contents were stirred at 20° C. for 1 hour. The contents were filtered, washed with MTBE (0.5 L), and pulled dry. The filtrate was dried over magnesium sulphate and filtered. The dry filtrate was concentrated via rotavapor to give an amber oil=324.5 g: bath temperature=40° C. HPLC: 98.0% stage 6. NMR assay (CDCl₃): 97%. Active yield=314.8 g (82% yield).

### Example 6

### Stage 7—Preparation of (R)-1-(benzo[d][1,3]dioxol-5-yl)-N-methylpropan-2-amine hydrochloride (7)

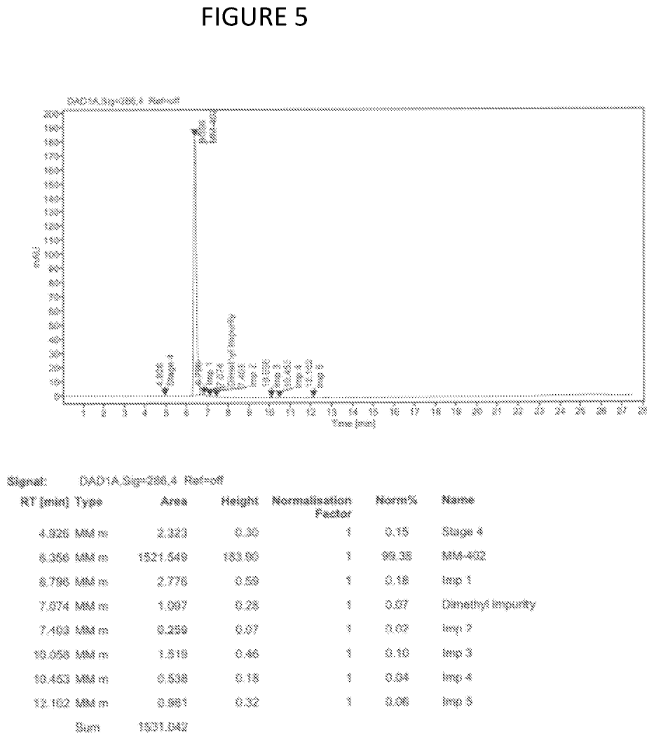

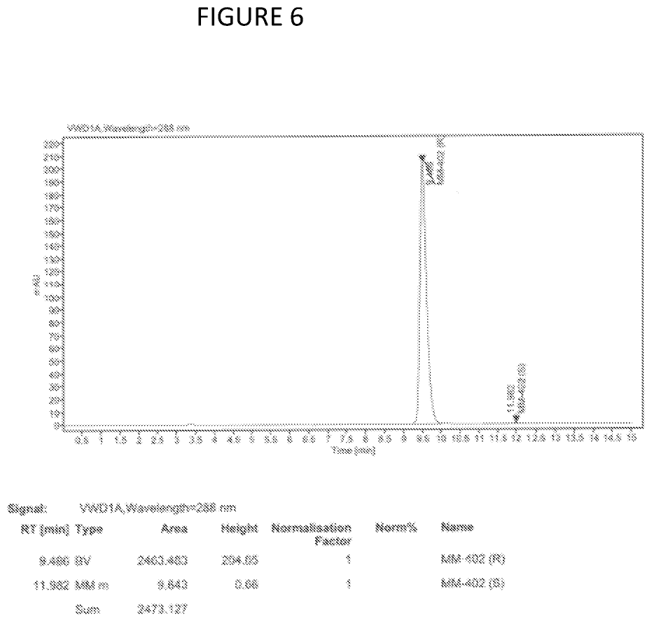

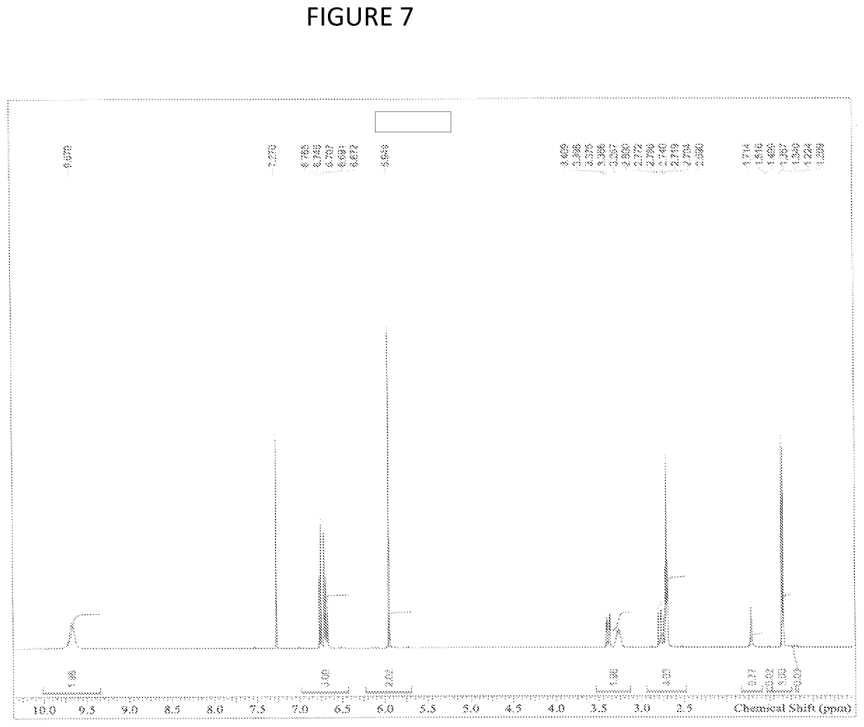

To a reactor were 6 (1227.1 g, 95% activity, 6.09 mol, 1 eq) and IPA (9.47 L). 5-6 M HCl in IPA (1023 ml, 5.77 mol, 1 eq) was charged dropwise. The resulting solids were isolated by filtration, washed with the mother the liquors and then IPA (300 ml), and dried under vacuum to give a white solid. Dry mass=1231 g. HPLC: 99.4% (FIG. 5). Chiral purity: 99.6% (FIG. 6), 99.2% e.e. ¹H NMR (FIG. 7) and MS conform to the expected structure.

### Example 7

Recrystallization of R-MDMA HCl

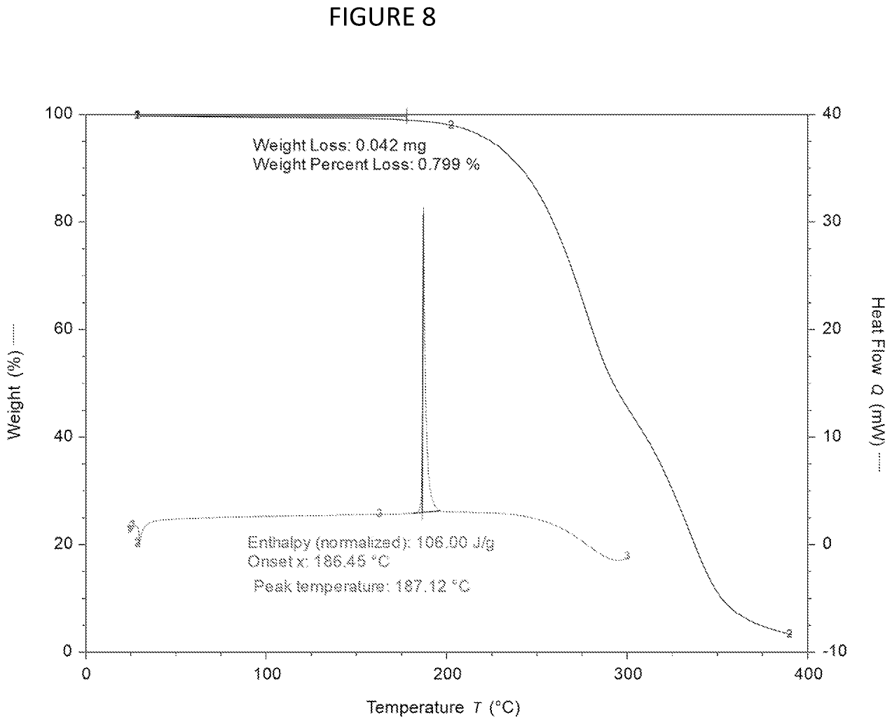

R-MDMA HCl (150 g) was charged into a flask. IPA/water (97:3, 7 vol, 1050 ml) was added and the suspension was heated to 70° C. The resulting solution was clarified into a preheated vessel and left to equilibrate at 70° C. for 30 minutes. The temperature was reduced to 55-60° C. and seeded with R-MDMA HCl Pattern A and left to develop for 15 minutes during which time a suspension was observed to form. The suspension was then cooled to 0° C. Two volumes of IPA were added. Following equilibration for about 2 hours, the suspension was filtered. The filter cake was washed with 1 vol cold IPA and the solids (damp mass 155 g) were dried in vacuo at 60° C. for 18 hours (dry mass 137.7 g, 91.8% yield). HPLC: 100.0%. Chiral purity: 100%, 100% e.e. ¹H NMR conforms to the expected structure. DSC and TGA thermographs of R-MDMA HCl Pattern A recrystallized from IPA/water are shown in FIG. 8.

### Example 8

Direct Preparation of Crystalline R-MDMA HCl Salt from R-MDMA Free Base

R-MDMA free base (active charge: 3.98 g) was charged into a 100 ml vessel. Water (0.84 ml) and IPA (22.39 ml) were added and the mixture was heated to 70° C. forming a light-yellow solution. Once at temperature, HCl in IPA (4.4M, 4.77 ml) was charged into the vessel over 1 hour and then the line was rinsed with IPA (0.5 vol, 2 ml). After the addition of the acid the solution was stirred for 30 minutes before being cooled to 58° C. The solution was seeded and left to develop. The resulting suspension was cooled to 0° C. IPA was added to the vessel (6 ml, 1.5 vols total) and left to stir for 2 hours. The solids were filtered and dried in vacuo for 18 hours at 60° C. A white solid (3.74 g, 79.4%) was obtained. HPLC: 100.0%. ¹H NMR conforms to the expected structure.

### Example 9

### S-MDMA Stage 1—Preparation of (R)-1-(benzo[d][1,3]dioxol-5-yl)propan-2-ol (8)

S-MDMA Stage 1 was performed on a 90 g scale following the process described in EXAMPLE 1 using R-propylene oxide in place of S-propylene oxide. The material was purified via distillation (in place of WFE distillation due to the smaller scale) to yield 62.32 g of a light blue oil (77% yield, target=78%) with an HPLC purity of 98.1%. ¹H NMR conforms to the expected structure. NMR assay=100%

### Example 10

### S-MDMA Stage 2—Preparation of (R)-1-(benzo[d][1,3]dioxol-5-yl)propan-2-yl methanesulfonate (9)

S-MDMA Stage 2 was performed on a 60 g scale following the process described in EXAMPLE 2 to afford 84.94 g of a brown oil with an HPLC purity of 97.8%. NMR assay=85% giving an active yield of 72.20 g with an 85% yield (target=80%).

### Example 11

### S-MDMA Stage 4—Preparation of (S)-1-(benzo[d][1,3]dioxol-5-yl)propan-2-amine (11)

S-MDMA Stages 3 and 4 were performed on an 84.9 g scale following the process described in EXAMPLE 3 to afford 45.81 g (88% yield, target=80%) of a yellow oil with an HPLC purity of 99.0% and a chiral purity of 99.0%, 98.0% e.e.

### Example 12

### S-MDMA Stage 5—Preparation of ethyl (S)-(1-(benzo[d][1,3]dioxol-5-yl)propan-2-yl)carbamate (12)

S-MDMA Stage 5 was performed on a 45.7 g scale following the process described in EXAMPLE 4 to afford 60.05 g (83% yield, target=92%) of a white solid with an HPLC purity of 99.6%. ¹H NMR conforms to the expected structure.

### Example 13

### S-MDMA Stage 6—Preparation of (S)-1-(benzo[d][1,3]dioxol-5-yl)-N-methylpropan-2-amine (13)

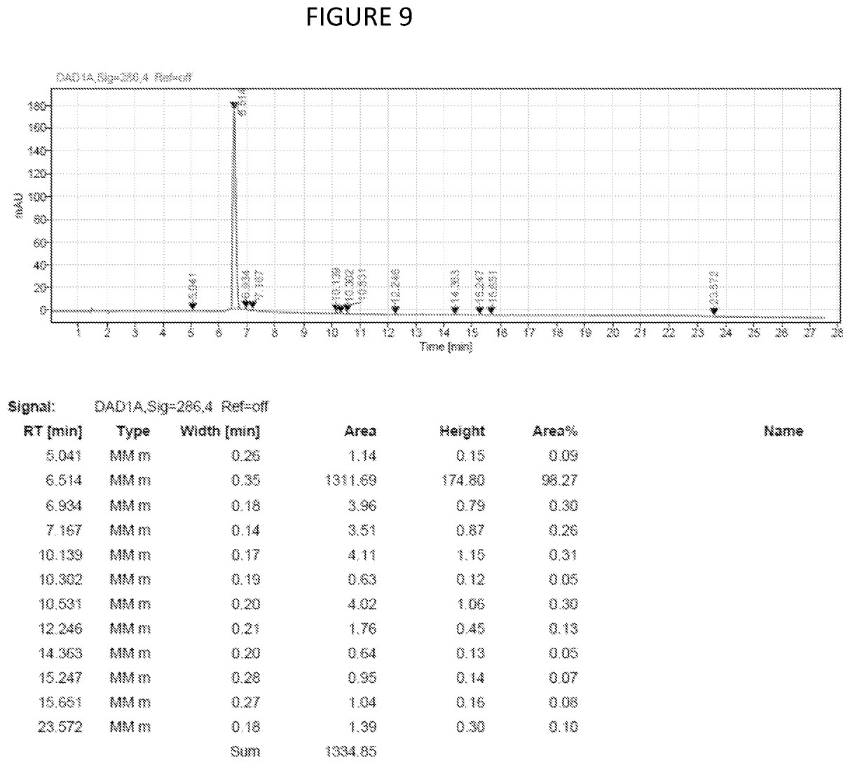

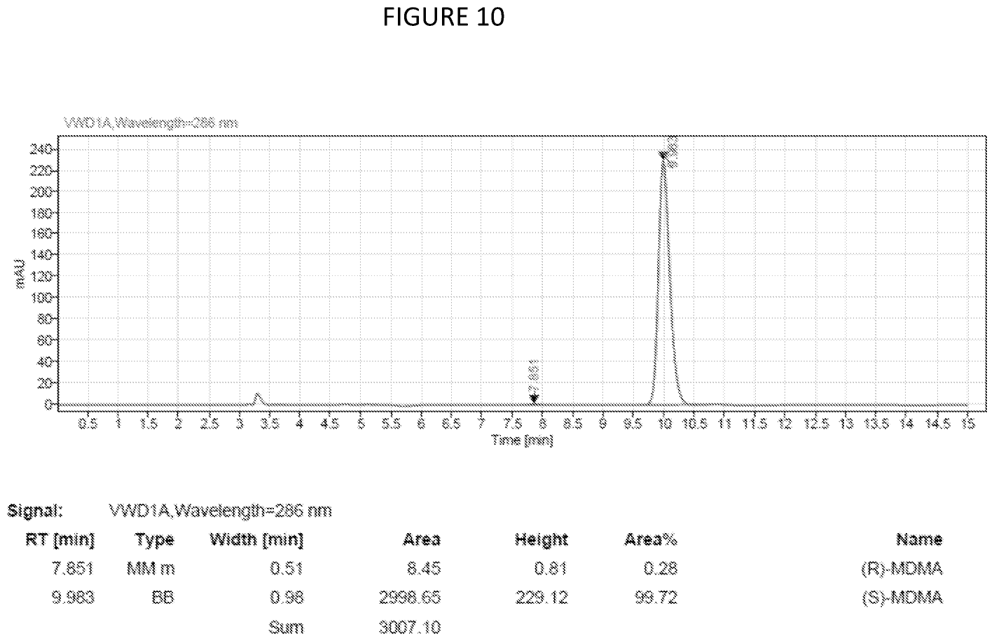

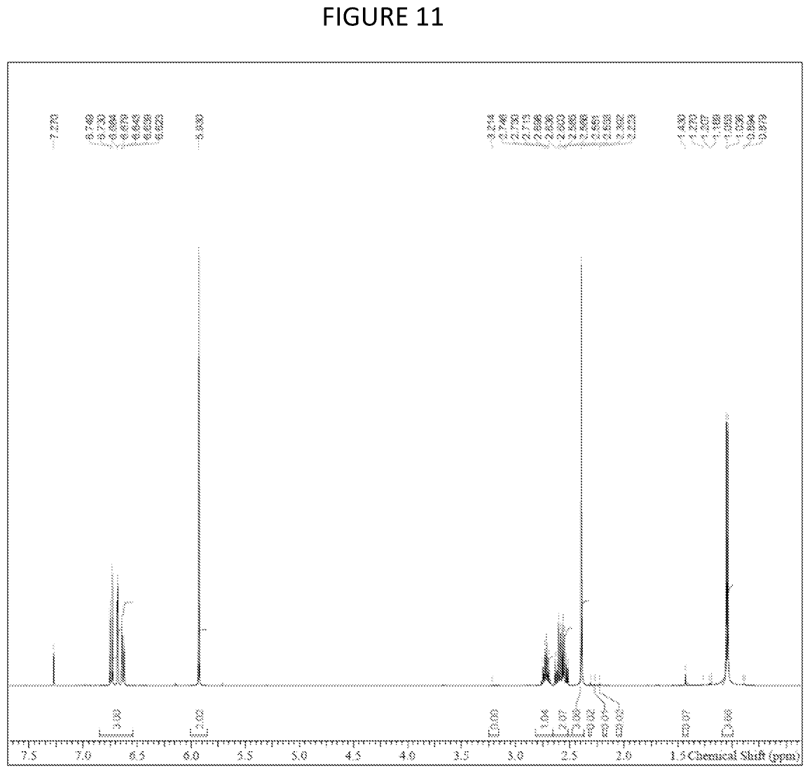

S-MDMA Stage 6 was performed on a 60.0 g scale following the process described in EXAMPLE 5 to afford 36.80 g as a pale yellow oil with an HPLC purity of 98.3% (FIG. 9) and a chiral purity of 99.7% (99.4% e.e., FIG. 10). ¹H NMR (FIG. 11) and MS conform to the expected structure. S-MDMA Free base 13 can be converted to a S-MDMA salt such as the hydrochloride (14) following the processes described in EXAMPLE 6 through EXAMPLE 8.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of manufacturing R-MDMA, by the steps shown in FIG. 3, including the steps of:
forming a Grignard reagent from 5-bromobenzodioxole;
treating the Grignard reagent with S-propylene oxide to form chirally pure alcohol (S)-1-(benzo[d][1,3]dioxol-5-yl)propan-2-ol (1);
activating the alcohol as mesylate (S)-1-(benzo[d][1,3]dioxol-5-yl)propan-2-yl methanesulfonate (2);
converting the mesylate to chirally pure azide ((R)-5-(2-azidopropyl)benzo[d][1,3]dioxole) (3);
reducing the azide to amine (R)-1-(benzo[d][1,3]dioxol-5-yl)propan-2-amine (4);
protecting the amine as ethyl (R)-(1-(benzo[d][1,3]dioxol-5-yl)propan-2-yl)carbamate (5);
reducing the protected amine to yield R-MDMA free base ((R)-1-(benzo[d][1,3]dioxol-5-yl)-N-methylpropan-2-amine) (6); and
treating the free base with an acid to form a salt of (R)-1-(benzo[d][1,3]dioxol-5-yl)-N-methylpropan-2-amine in >99% enantiomeric excess.

2. The method of claim 1, wherein said protecting step is further defined as protecting the amine with a compound chosen from the group consisting of di-tert-butyl dicarbonate and ethyl chloroformate.

3. The method of claim 1, wherein said reducing step is further defined as reducing the protected amine 5 with LiAlH₄.

4. The method of claim 1, wherein said salt formed in said treating step is chosen from the group consisting of hydrochloride, hydrobromide, maleate, L-malate, D-tartrate, meso-tartrate, citrate, phosphate, naphthylene-1,5-disulphonate, fumarate, sulfate, mesylate, acetate, and oxalate.

5. The method of claim 1, wherein said method provides at least 30% overall yield of R-MDMA.

6. The method of claim 1, further including the step of converting the amine 4 to a salt for purification or storage.

7. The method of claim 6, wherein the salt is (R)-1-(benzo[d][1,3]dioxol-5-yl)-N-methylpropan-2-amine hydrochloride (7).

8. A method of manufacturing S-MDMA, by the steps shown in FIG. 4, including the steps of:
forming a Grignard reagent from 5-bromobenzodioxole;
treating the Grignard reagent with R-propylene oxide to form chirally pure (R)-1-(benzo[d][1,3]dioxol-5-yl)propan-2-ol (8);
activating the alcohol (8) as mesylate (R)-1-(benzo[d][1,3]dioxol-5-yl)propan-2-yl methanesulfonate (9);
converting the mesylate (9) to chirally pure azide ((S)-5-(2-azidopropyl)benzo[d][1,3]dioxole) (10);
reducing the azide (10) to amine (S)-1-(benzo[d][1,3]dioxol-5-yl)propan-2-amine (11);
protecting the amine (11) with di-tert-butyl dicarbonate as ethyl (S)-(1-(benzo[d][1,3]dioxol-5-yl)propan-2-yl)carbamate (12);
reducing the protected amine (12) to yield S-MDMA free base ((S)-1-(benzo[d][1,3]dioxol-5-yl)-N-methylpropan-2-amine) (13); and
treating with an acid to form a salt of (S)-1-(benzo[d][1,3]dioxol-5-yl)-N-methylpropan-2-amine in >99% enantiomeric excess.

9. The method of claim 8, wherein said protecting step is further defined as protecting the amine with a compound chosen from the group consisting of di-tert-butyl dicarbonate and ethyl chloroformate.

10. The method of claim 8, wherein said reducing step is further defined as reducing the protected amine 5 with LiAlH₄.

11. The method of claim 8, wherein said salt formed in said treating step is chosen from the group consisting of hydrochloride, hydrobromide, maleate, L-malate, D-tartrate, meso-tartrate, citrate, phosphate, naphthylene-1,5-disulphonate, fumarate, sulfate, mesylate, acetate, and oxalate.

12. The method of claim 8, wherein said method provides at least 30% overall yield of S-MDMA.

13. The method of claim 8, further including the step of converting the amine 11 to a salt for purification or storage.

14. The method of claim 13, wherein the salt is (S)-1-(benzo[d][1,3]dioxol-5-yl)-N-methylpropan-2-amine hydrochloride (14).
